(19) United States
(12) Patent Application Publication
(10) Pub. No.: US 2011/0098488 A1
(43) Pub. Date: Apr. 28, 2011

(54) Process for Oxidizing Organic Substrates By Means of Singlet Oxygen Using a Modified Molybdate LDH Catalyst

(76) Inventors: Dirk De Vos, Holsbeek (BE); Pierre Jacobs, Goojk (BE); Joos Wahlen, Berlaar (BE); Paul Alsters, Maastricht (NL); Walther Jary, Steinbach am Attersee (AT)

(21) Appl. No.: 11/918,131

(22) PCT Filed: Mar. 18, 2006
(86) PCT No.: PCT/EP2006/002510

§ 371 Date: Apr. 2, 2008

(30) Foreign Application Priority Data
Apr. 13, 2005 (AT) A 620/2005

(51) Int. Cl. C07C 409/04; C07C 409/18

(52) U.S. Cl. 549/363; 568/567

(57) ABSTRACT

Oxidation of organic substrates by means of singlet oxygen, in which organic substrates which react with ¹O₂ are admixed with 10-70% H₂O₂ in an organic solvent in the presence of a molybdate LDH catalyst modified by ethylene glycol, polyethylene glycol or polyol, and the catalytic decomposition of H₂O₂ to water and ¹O₂ is then followed by the oxidation to the corresponding oxidation products, and also modified molybdate LDH catalysts.

Process for oxidizing organic substrates by means of singlet oxygen using a modified molybdate LDH catalyst.

The invention relates to a process for oxidizing organic substrates by means of singlet oxygen using a modified molybdate LDH catalyst, and also the modified molybdate LDH catalyst itself.

The sole singlet oxygen oxidation (¹O₂-Ox) which is currently performed industrially is the photochemical ¹O₂-Ox in which the ¹O₂ is generated by a photochemical route. The disadvantage of this process results from the high costs of the photochemical devices required, and also restricted lifetime. The lamps required are degenerated relatively rapidly during the oxidation as a result of soiling of the glass surface. This process is also unsuitable for colored substrates. The process is actually only suitable for fine chemicals which are prepared on a relatively small scale (La Chimica e l'lndustria, 1982, Vol. 64, page 156).

For this reason, attempts have been made to find other process variants for the ¹O₂-Ox which are suitable for the ¹O₂-Ox of water-insoluble hydrophobic organic substrates.

J. Am. Chem. Soc., 1968, 90, 975 describes, for example, the classical “dark” ¹O₂-Ox in which ¹O₂ is generated not photochemically but rather chemically. In this case, hydrophobic substrates are oxidized by means of a hypochlorite/H₂O₂ system in a solvent mixture composed of water and organic solvent. However, this process has found only a few synthetic applications, since many substrates are only sparingly soluble in the medium required. Moreover, the usability is quite restricted owing to side reactions between hypochlorite and substrate or solvent. Also, a large portion of the ¹O₂ is deactivated in the gas phase. Furthermore, this process is unsuitable for the industrial scale, since there is addition of the hypochlorite to H₂O₂ in the organic medium and a large excess of H₂O₂ is required to suppress the side reaction of substrate with hypochlorite. An additional disadvantage arises by virtue of the occurrence of stoichiometric amounts of salt.

One variant of the “dark” ¹O₂-Ox, which is not based on hypochlorite and is thus intended to partly avoid the above disadvantages, is known, for example, from J. Org. Chem., 1989, 54, 726 or J. Mol. Cat., 1997, 117, 439, according to which some water-soluble organic substrates are oxidized with H₂O₂ and a molybdate catalyst in water as a solvent. According to Membrane Lipid Oxid. Vol. 11, 1991, 65 the ¹O₂-Ox of water-insoluble organic substrates with the molybdate/H₂O₂ system is difficult, since it was assumed that none of the customary solvents is suitable for maintaining the molybdate-catalyzed disproportionation of H₂O₂ in water and ¹O₂. However, the use of molybdenum catalysts also entails other disadvantages, for instance difficulty of recycling or environmental pollution.

Various literature sources, for example Adv. Synth. Catal. 2004, 346, 152-164, Chem. Commun., 1998, 267 or Chem. Eur. J. 2001, 7, 2547, disclose the use of molybdate LDH catalysts for singlet oxygen oxidation, but these do not have satisfactory selectivity and do not afford satisfactory yields.

A further means of chemically generating ¹O₂ is, for example, the heating of triphenyl phosphite ozonide which is obtained from triphenyl phosphite and ozone. However, this method is, as described, for instance, in J. Org. Chem., Vol. 67, No 8, 2002, page 2418, employed only for mechanistic studies, since triphenyl phosphite is an expensive and additionally dangerous chemical.

In the base-catalyzed disproportionation of peracids, not only ¹O₂ but also further reactive compounds are formed, which lead to by-products.

It is accordingly an object of the present invention to enable the oxidation of organic substrates by means of singlet oxygen (¹O₂) with avoidance of molybdenum-containing wastewaters, and also to find a catalytic system with high activity and selectivity therefor.

Unexpectedly, this object is achieved by the use of a modified, heterogeneous molybdate LDH catalyst.

The present invention accordingly provides for the oxidation of organic substrates by means of singlet oxygen, which comprises admixing organic substrates which react with ¹O₂ with 10-70% H₂O₂ in an organic solvent in the presence of a molybdate LDH catalyst modified by ethylene glycol, polyethylene glycol or polyols, and the catalytic decomposition of H₂O₂ to water and ¹O₂ then being followed by the oxidation to the corresponding oxidation products.

In the process according to the invention, organic substrates are oxidized by means of singlet oxygen.

The organic substrates used which react with ¹O₂ may be the following compounds: olefins which contain one or more, i.e. up to 10, preferably up to 6, more preferably up to 4 C═C double bonds; electron-rich aromatics such as C₆-C₅₀, preferably up to C₃₀, more preferably up to C₂₀ phenols, polyalkylbenzenes, polyalkoxybenzenes; polycyclic aromatics having from 2 to 10, preferably up to 6, more preferably up to 4 aromatic rings; sulfides, for instance alkyl sulfides, alkenyl sulfides, aryl sulfides, which are either mono- or disubstituted on the sulfur atom, and also heterocycles having an oxygen, nitrogen or sulfur atom in the ring, for example C₄-C₅₀, preferably up to C₃₀, more preferably up to C₂₀ furans, C₄-C₅₀, preferably up to C₃₀, more preferably up to C₂₀ pyrroles, C₄-C₆₀, preferably up to C₃₀, more preferably up to C₂₀ thiophenes.

The substrates may have one or more substituents, such as halogen (F, Cl, Br, I), cyanide, carbonyl groups, hydroxyl groups, C₁-C₅₀, preferably up to C₃₀, more preferably up to C₂₀ alkoxy groups, C₁-C₅₀, preferably up to C₃₀, more preferably up to C₂₀ alkyl groups, C₆-C₅₀, preferably up to C₃₀, more preferably up to C₂₀ aryl groups, C₂-C₅₀, preferably up to C₃₀, more preferably up to C₂₀ alkenyl groups, C₂-C₅₀, preferably up to C₃₀, more preferably up to C₂₀ alkynyl groups, carboxylic acid groups, ester groups, amide groups, amino groups, nitro groups, silyl groups, silyloxy groups, sulfone groups, sulfoxide groups, etc. The substrates may also be substituted by one or more NR1R2 radicals in which R1 and R2 may be the same or different and are each H; C₁-C₅₀, preferably up to C₃₀, more preferably up to C₂₀ alkyl; formyl; C₂-C₅₀, preferably up to C₃₀, more preferably up to C₂₀ acyl; C₇-C₅₀, preferably up to C₃₀, more preferably up to C₂₀ benzoyl, where R1 and R2 may also together form a ring, for example in a phthalimido group.

Examples of suitable substrates are: 2-butene; isobutene; 2-methyl-1-butene; 2-hexene; 1,3-butadiene; 2,3-dimethylbutene; Δ^{9,10}-octalin, 2-phthalimido-4-methyl-3-pentene; 2,3-dimethyl-1,3-butadiene; 2,4-hexadiene; 2-chloro-4-methyl-3-pentene; 2-bromo-4-methyl-3-pentene; 1-trimethylsilylcyclohexene; 2,3-dimethyl-2-butenyl-para-tolylsulfone; 2,3-dimethyl-2-butenyl-para-tolyl sulfoxide; N-cyclohexenylmorpholine; 2-methyl-2-norbornene; terpinolene; α-pinene; β-pinene; β-citronellol; ocimene; citronellol; geraniol; farnesol; terpinene; limonene; trans-2,3-dimethylacrylic acid; α-terpinene; isoprene; cyclopentadiene; 1,4-diphenylbutadiene; 2-ethoxybutadiene; 1,1′-dicyclohexenyl; cholesterol; ergosterol acetate; 5-chloro-1,3-cyclohexadiene; 3-methyl-2-buten-1-ol; 3,5,5-trimethylcyclohex-2-en-1-ol; phenol, 1,2,4-trimethoxybenzene, 2,3,6-trimethylphenol, 2,4,6-trimethylphenol, 1,4-dimethylnaphthalene, furan, furfuryl alcohol, furfural, 2,5-dimethylfuran, isobenzofuran, dibenzyl sulfide, 2-methyl-5-tert-butylphenyl sulfide, etc.

The corresponding oxidation product is obtained from the substrates by the inventive oxidation. From alkenes, (polycyclic) aromatics or heteroaromatics, especially hydroperoxides or peroxides are obtained and can react further under the reaction conditions to give alcohols, epoxides, acetals or carbonyl compounds such as ketones, aldehydes, carboxylic acids or esters when the hydroperoxide or the peroxide is unstable.

The inventive oxidation is effected in an organic solvent.

Suitable solvents are C₁-C₈ alcohols such as methanol, ethanol, propanol, i-propanol, butanol, i-butanol, n-butanol, tert-butanol, ethylene glycol, propylene glycol, acetone, 1,4-dioxane, tetrahydrofuran, formamide, N-methylformamide, dimethylformamide, sulfolane, propylene carbonate and mixtures thereof. Preference is given to using methanol, ethanol, propanol, i-propanol, ethylene glycol, propylene glycol, acetone, formamide, N-methylformamide or dimethylformamide, particular preference to using methanol, ethanol, ethylene glycol, propylene glycol, formamide or dimethyl formamide as solvents.

If appropriate, up to 25% of water may be added to the organic solvent. However, the addition of water does not bring any advantages for the reaction. Preference is therefore given to not adding any water.

According to the invention, the heterogeneous catalyst added to the solvent-substrate mixture is a molybdate LDH catalyst modified by ethylene glycol, polyethylene glycol or by polyols (e.g. glycerol).

Unmodified molybdate(Mo) LDH catalysts (LDH . . . layered double hydroxides) are already prior art and are described, for example, in Adv. Synth. Catal. 2004, 346, 152-164.

The unmodified Mo LDH catalysts are prepared, for example, according to the prior art (for example Chem. Eur. J. 2001, 7, No. 12, P. 2556) by reacting magnesium nitrate hydrates and aluminum nitrate hydrates in the presence of NaOH and subsequent addition of Na₂MoO₄.2H₂O.

In the case of these catalysts, the reaction product from the magnesium nitrate hydrates and aluminum nitrate hydrates forms the support material which, on completion of reaction, can first be isolated or can be treated directly with the molybdenum compound to exchange the nitrate groups for the (MoO₄)²⁻.

The molar Mg/Al ratio in these catalysts may vary from 10 to 2. Preference is given to an Mg/Al ratio of 2:1.

The amount of molybdate compound used depends upon the desired loading of the support with molybdenum and may vary from 0.002 mmol Mo/g of catalyst to 2 mol Mo/g of catalyst.

In the modified Mo LDH catalysts used in accordance with the invention, an Mo LDH catalyst obtained according to the prior art is suspended in ethylene glycol, polyethylene glycol or polyol and kept in suspension for from a few hours up to several days at elevated temperature, preferably at from 60 to 100° C., more preferably at from 75 to 85° C.

After the treatment with EG, PEG or polyol, the now modified Mo LDH catalyst is isolated from the suspension, dried under reduced pressure and can then be used in accordance with the invention.

These Mo LDH catalysts modified by EG, PEG or polyol are novel and therefore likewise form part of the subject matter of the present invention.

The amount of catalyst used depends upon the substrate used and is between 0.001 and 50 mol %, preferably between 0.1 and 10 mol %.

Subsequently, 10-70%, preferably 40-50% H₂O₂, is added. H₂O₂ is preferably added slowly or in portions to the reaction mixture composed of solvent, substrate and catalyst, in the course of which the reaction mixture is preferably stirred.

The consumption of H₂O₂ in the process according to the invention is dependent upon the substrate used. For reactive substrates, preferably from 2 to 3 equivalents of H₂O₂ are required, while less reactive substrates are preferably reacted with from 3 to 10 equivalents of H₂O₂

The reaction temperature is between −20 and +80° C., preferably between 15 and 60° C.

The reaction progress can be monitored by means of UV spectroscopy or by means of HPLC.

After the reaction has ended, the reaction mixture is worked up.

After filtering off the catalyst, the reaction solution which comprises the oxidation product is worked up by customary methods, for instance extraction, drying and isolation of the oxidation product, for example by column chromatography.

The catalyst filtered off in accordance with the invention can then be used without further purification or drying for further oxidations.

The process according to the invention generates ¹O₂ in a simple and efficient manner.

The process according to the invention affords the desired end products in high yields of up to 100% with high purity.

The process according to the invention is notable for the simple process which is ideally suited to the industrial scale, since it can be effected in simple multipurpose plants and with simple workup steps, and can be employed for a broad spectrum of substrates. A further advantage is the repeated reusability of the inventive catalyst.

### EXAMPLE 1

### Synthesis and Characterization of Molybdate LDH (LDH=Layered Double Hydroxide) Catalysts

### a) Preparation of the Support Material

A 1 l three-neck flask was charged with 100 ml of distilled water and the pH was adjusted to 10 with 1 M sodium hydroxide solution under a nitrogen atmosphere. 120 ml of a 0.333 M Al(NO₃)₃₋₆H₂O solution and 120 ml of a 0.667 M Mg(NO₃)₂.6H₂O solution were then introduced simultaneously into the flask with good stirring (metering rate 100 ml/h). During the metered addition of the two salt solutions, the pH was kept constant at 10 (by means of metering in a 1 M NaOH solution by means of a peristaltic pump). Once the salt solutions had been metered in, the suspension was stirred at room temperature for a further 22 hours. Thereafter, the precipitate formed was centrifuged, washed and centrifuged again. The washings were carried out three times (washing water required 3×400 ml).

The precipitate thus obtained was then dried by means of freeze-drying.

Yield (dry): 10 g of {[Mg/Al]LDH²⁺(NO₃)²⁻} catalyst support, white powder

### b) Application of (MoO₄)²⁻ to the Catalyst Surface by Exchange of (NO₃)²⁻

10 g of [Mg/Al]LDH(NO₃)⁻ catalyst support (Mg/Al=2) were added to a 2 mM Na₂MoO₄.2H₂O solution in water (volume 1 liter). The suspension was stirred at room temperature under inert gas atmosphere for a further 12 hours. The precipitate was centrifuged and washed twice with deionized water (400 ml per washing operation). The precipitate thus obtained was then dried by means of freeze-drying.

The Mo content was determined by means of ICP-AES which gave 0.02 mmol of Mo per gram of catalyst support.

Yield (dry): 9.8 g of {[Mg/Al]LDH²⁺(MoO₄)²⁻} catalyst, white powder

c) Catalyst Preparation without Isolation of the Support Material

A 1 l three-neck flask was charged with 100 ml of distilled water and the pH was adjusted to 10 with 1 M sodium hydroxide solution under a nitrogen atmosphere. 120 ml of a 0.333 M Al(NO₃)₃.6H₂O solution and 120 ml of a 0.667 M Mg(NO₃)₂.6H₂O solution were then introduced simultaneously into the flask with good stirring (metering rate 100 ml/h). During the metered addition of the two salt solutions, the pH was kept constant at 10 (by means of metering in a 1 M NaOH solution by means of a peristaltic pump). Once the salt solutions had been metered in, the suspension was stirred at room temperature for a further 22 hours.

Thereafter, a 2 mM Na₂MoO₄.2H₂O solution in water (volume 1 liter) was added to the catalyst support suspension. The suspension was stirred at room temperature under an inert gas atmosphere for a further 12 hours. The precipitate was filtered off and dried at 60° C. under reduced pressure.

Yield (dry): 11 g of {[Mg/Al]LDH²⁺(MoO₄)²⁻} catalyst, white powder

The Mo content was determined by means of ICP-AES which gave 0.02 mmol of Mo per gram of catalyst support.

### EXAMPLE 2

### Preparation of an Mo LDH Catalyst Modified by Ethylene Glycol

An Mo LDH catalyst prepared according to example 1 was suspended in 10 times the amount of ethylene glycol and kept in suspension at 80° C. for 12 hours. Subsequently, the mixture is filtered and the catalyst is dried under reduced pressure.

The Mo content was determined by means of ICP-AES which gave 0.02 mmol of Mo per gram of catalyst support. The catalyst was characterized by means of FTIR.

### EXAMPLES 3-12

### Use Examples of the Catalyst

A general procedure for the oxidation of olefinic compounds was as follows:

In a 25 ml round-bottom flask, 0.25 g of Mo LDH EG (Mg/Al=2; 0.2 mmol of Mo/g), 5 mmol of olefin and 5 ml of N,N-dimethylformamide were mixed at 25° C. with good stirring, and H₂O₂(50% by weight) was added in portions of 2.5 mmol per portion. In the course of this, the color of the initially white suspension became yellowish to orange. The reaction progress was observed by means of GC.

The results for the oxidation of olefins are compiled in the table which follows.

| Substrate | Product Distribution | Conversion | Selectivity |
|---|---|---|---|
| Example | [%] | [%] | [%] |
| 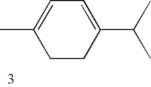 | 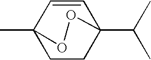 | 98 | 86 |
| 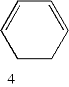 | 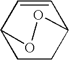 | 92 | 82 |
| 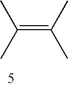 | 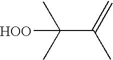 | 99 | 99 |
| 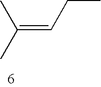 | 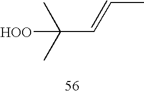 | 99 | 99 |
| | 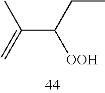 | | |
| 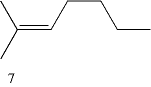 | 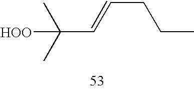 | 97 | 99 |
| | 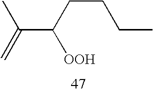 | | |
| 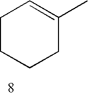 | 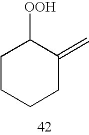 | 98 | 99 |
| | 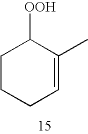 | | |
| | 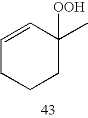 | | |
| 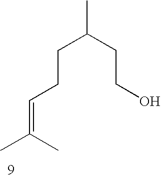 | 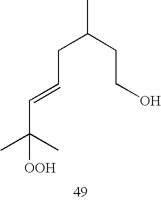 | 96 | 99 |
| | 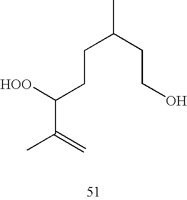 | | |
| 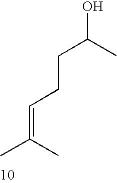 | 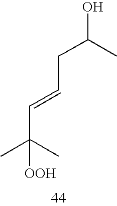 | 99 | 99 |
| | 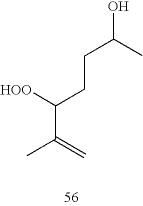 | | |
| 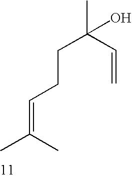 | 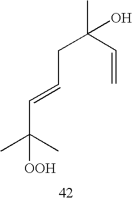 | 99 | 99 |
| | 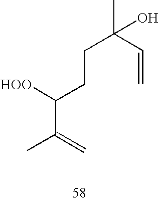 | | |
| 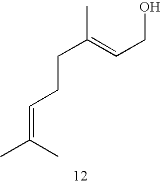 | 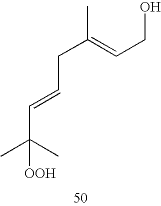 | 92 | 75 ^{[a]} |
| | 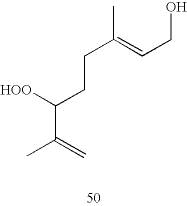 | | |

^{[a]} Limited peroxide formation of the 2,3 double bond was observed.

### EXAMPLES 13-18

### Use Examples of the Catalyst

A general procedure for the oxidation of allylic alcohols was as follows:

In a 25 ml round-bottom flask, 0.1 g of Mo LDH EG (Mg/Al=2; 0.2 mmol of Mo/g), 2 mmol of allyl alcohol and 2 ml of N,N-dimethylformamide were mixed at 25° C. with good stirring and H₂O₂(50% by weight) was added in portions of 0.5 mmol per portion. The reaction progress was observed by means of GC.

Results for the oxidation of allylic alcohols are shown in the next table.

| Substrate | Product Distribution | Conversion | Selectivity |
|---|---|---|---|
| Example | [%] | [%] | [%] |
| 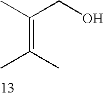 | 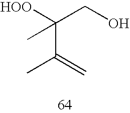 | 99 | 99 |
| | 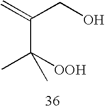 | | |
| 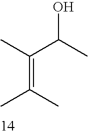 | 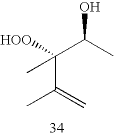 | 99 | 94 |
| | 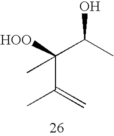 | | |
| | 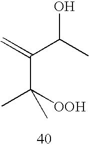 | | |
| 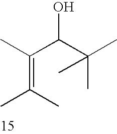 | 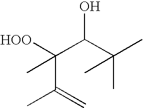 | 99 | 99 |
| 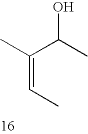 | 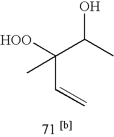 | 97 | 99 |
| | 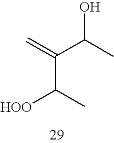 | | |
| 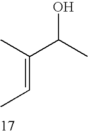 | 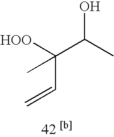 | 99 | 99 |
| | 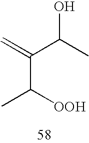 | | |
| 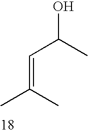 | 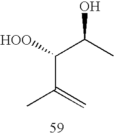 | 88 | 98 |
| | 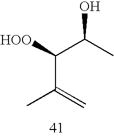 | | |

^{[b]} GC-incomplete separation of the diastereomers.

### EXAMPLE 19

### Examples of the Use of Other Solvents

Other solvents were also used instead of N,N-dimethylformamide for the peroxide generation. The results are shown in the graph which follows.

The model substrate used was citronellol.

Reaction conditions: 0.5 g of Mo LDH EG (Mg/Al=2; 0.2 mmol of Mo/g, treated with EG for 5 days at 80° C.), 10 mmol of citronellol, 40 mmol of H₂O₂ (50% by wt.) added in 5 mmol portions, 10 ml of solvent, 25° C.

### EXAMPLE 20

### Comparative Experiment

### Advantage of the Mo LDH EG Catalyst Over the Unmodified Mo LDH Catalyst

The effect of the EG modification of the Mo LDH catalyst surface on the peroxidation of citronellol is shown in FIG. 2. The data show the total time for hydrogen peroxide disproportionation.

Reaction conditions: 0.5 g of Mo LDH or Mo LDH EG (Mg/Al=2, 0.2 mmol of Mo/g, ethylene glycol for 12 h at 80° C.), 10 mmol of citronellol, 40 mmol of H₂O₂ (50% by wt.) added in 5 mmol portions, 10 ml DMF, 25° C. Selectivity >99% in both cases.

### EXAMPLE 23

### Oxidation of Linalool

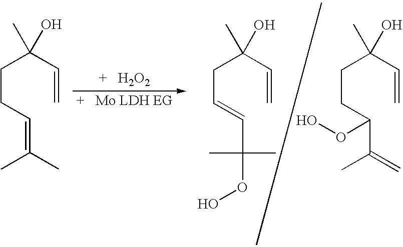

A 2000 liter jacketed vessel was charged with 1400 ml of methanol, and 1 mol % of catalyst (based on linalool) and 200 g (240 ml) of linalool were added. With good stirring, 358.8 g (300 ml) of 50% hydrogen peroxide were added at 25° C. within 8 hours. The reaction progress was monitored by means of GC.

Conversion: >95% (Table)

| Sample Number | Linalool | Dimethyloctadienediol | By-products |
|---|---|---|---|
| 1 | 98.18 | 0.00 | 1.82 |
| 2 | 88.93 | 9.00 | 2.07 |
| 3 | 77.28 | 20.75 | 1.97 |
| 4 | 61.75 | 35.82 | 2.43 |
| 5 | 47.49 | 49.22 | 3.29 |
| 6 | 33.78 | 62.88 | 3.34 |
| 7 | 21.07 | 75.44 | 3.49 |
| 8 | 11.09 | 85.15 | 3.76 |
| 9 | 5.28 | 88.46 | 6.26 |
| 10 | 4.34 | 92.66 | 3.00 |
| 11 | 4.34 | 95.58 | 0.08 |
| 12 | 3.77 | 92.97 | 3.26 |
| 13 | 3.40 | 94.42 | 2.18 |

What is claimed is:

1. An oxidation of organic substrates by means of singlet oxygen, which comprises admixing organic substrates which react with ¹O₂ with 10-70% H₂O₂ in an organic solvent in the presence of a molybdate LDH catalyst modified by ethylene glycol, polyethylene glycol or polyol, and the catalytic decomposition of H₂O₂ to water and ¹O₂ then being followed by the oxidation to the corresponding oxidation products.

2. The process as claimed in claim 1, wherein the substrates used which react with ¹O₂ are olefins which contain from 1 to 10 C═C double bonds; C₆-C₅₀ phenols, polyalkylbenzenes, polyalkoxybenzenes; polycyclic aromatics having from 2 to 10 aromatic rings; alkyl sulfides, alkenyl sulfides, aryl sulfides, which are either mono- or disubstituted on the sulfur atom, and also C₄-C₆₀ heterocycles having an oxygen, nitrogen or sulfur atom in the ring, which may be unsubstituted or mono- or polysubstituted by halogens, cyanide, carbonyl groups, hydroxyl groups, C₁-C₅₀ alkoxy groups, C₁-C₅₀ alkyl groups, C₆-C₅₀ aryl groups, C₂-C₅₀ alkenyl groups, C₂-C₅₀ alkynyl groups, carboxylic acid groups, ester groups, amide groups, amino groups, nitro groups, silyl groups, silyloxy groups, sulfone groups, sulfoxide groups, or by one or more NR¹R² radicals in which R₁ and R₂ may be the same or different and may be H; C₁-C₅₀ alkyl; formyl; C₂-C₅₀ acyl; C₇-C₅₀ benzoyl, where R¹ and R² may also together form a ring.

3. The process as claimed in claim 1, wherein the solvents used are C₁-C₈ alcohols, acetone, 1,4-dioxane, tetrahydrofuran, formamide, N-methylformamide, dimethylformamide, sulfolane, propylene carbonate or mixtures thereof.

4. The process as claimed in claim 3, wherein the solvents used are methanol, ethanol, propanol, i-propanol, ethylene glycol, propylene glycol, acetone, form amide, N-methylformamide or dimethylformamide.

5. The process as claimed in claim 1, wherein from 0.001 to 50 mol % of catalyst is used depending on the substrate.

6. The process as claimed in claim 1, wherein from 2 to 10 equivalents of H₂O₂ are used depending upon the substrate used.

7. The process as claimed in claim 1, wherein the reaction temperature is between −20 and +80° C.

8. The process as claimed in claim 1, wherein, after the reaction of the hydrophobic, organic substrates which react with ¹O₂ to give the corresponding oxidation products, the catalyst is removed on completion of reaction by simple filtering out of the reaction mixture and is then used for further oxidations.

9. A molybdate LDH catalyst which has been modified by ethylene glycol, polyethylene glycol or polyols.

10. A process for preparing catalysts as claimed in claim 9, which comprises suspending a molybdate LDH catalyst in ethylene glycol, polyethylene glycol or a polyol and keeping it in suspension at elevated temperature for from a few hours up to several days, then isolating and drying the modified catalyst.

11. The use of catalysts as claimed in claim 9 for generating singlet oxygen from H₂O₂.
